(19) United States
(12) Patent Application Publication
(10) Pub. No.: US 2021/0219848 A1
(43) Pub. Date: Jul. 22, 2021

(54) METHOD AND APPARATUS FOR MEASURING ROBUST PULSE RATE AND RESPIRATORY RATE USING FACIAL IMAGES

(71) Applicant: Research & Business Foundation Sungkyunkwan University, Suwon-si (KR)

(72) Inventors: Kwang Seok HONG, Suwon-si (KR); Jin Soo PARK, Suwon-si (KR); Jae Gap LEE, Suwon-si (KR)

(73) Assignee: Research & Business Foundation Sungkyunkwan University, Suwon-si (KR)

(21) Appl. No.: 17/154,112

(22) Filed: Jan. 21, 2021

(30) Foreign Application Priority Data
Jan. 21, 2020 (KR) 10-2020-0008182

(51) Int. Cl. A61B 5/0205; A61B 5/00; A61B 5/103; H04N 1/60

(52) U.S. Cl. CPC A61B 5/0205; A61B 5/7257; A61B 5/02416; A61B 5/1032; H04N 1/6016; A61B 5/7246

(57) ABSTRACT

A pulse rate and respiratory rate measuring method includes acquiring a facial image comprising a face of a user, calculating a color signal from a predetermined number of skin regions of interest in the face of the user in the facial image, calculating a pulse wave signal or a respiratory signal by converting an extracted signal into a time domain after extracting a signal in a frequency band corresponding to pulse or respiration in frequency data obtained by converting the color signal into a frequency domain, and measuring a pulse rate or a respiratory rate corresponding to the frequency band based on peak interval information in the pulse wave signal or the respiratory signal.

### CROSS-REFERENCE TO RELATED APPLICATIONS

This application claims the benefit under 35 USC 119(a) of Korean Patent Application No. 10-2020-0008182 filed on Jan. 21, 2020, in the Korean Intellectual Property Office, the entire disclosure of which is incorporated herein by reference for all purposes.

### BACKGROUND

### 1. Field

The present disclosure relates to a method and an apparatus for measuring a biosignal such as a pulse rate and a respiratory rate using facial images of a user.

### 2. Description of the Related Art

Both tachycardia and bradycardia are signs of abnormal heart function, which appears when there are abnormalities of heart rate. Generally, when the heart rate per minute is 100 beats or more, it is called tachycardia, and when the heart rate per minute is 60 beats or less, it is called bradycardia. Recently, a technique of measuring a pulse in accordance with a change in a skin color using a body image has been developed in order to identify an abnormal sign of the heart functions anytime and anywhere.

Respiration is one of four vital signs, which are important signals indicating the state and function of the body, and is deeply related to heart rate variability (HRV), and is one element of important biosignals in relation to respiratory disorders of heart disease patients or newborn babies. Recently, a technique of measuring a respiratory rate of a subject in a non-contact method by measuring a respiratory rate using a non-contact image from a smart device equipped with a camera possessed by a user without installing an additional hardware module has been developed.

However, the pulse rate and the respiratory rate measured using RGB colors of a skin image according to the related art are highly affected by surrounding environments and the change in illumination so that in some cases, there is a big difference between a pulse rate measured using the contact type measurement equipment and a pulse rate and a respiratory rate estimated using an image. In order to solve this problem, recently, a technique of measuring a pulse rate and a respiratory rate in a non-contact method by applying fast Fourier transform (FFT) and bandpass filter (BPF) to a C_{g} color signal calculated by converting an RGB color system into a YC_{g}Cₒ color system has been developed. However, even though this technique is used, there is still an error from the pulse measured using the contact type measurement equipment.

Accordingly, in order to improve the above-described problems and replace the biosignal measurement equipment, a method of capturing a facial image in a non-contact method using a normal camera, an IR camera, and a zoom camera possessed by a user without installing an additional hardware module and measuring a pulse rate and a respiratory rate with high precision using the same is necessary.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a pulse rate and respiratory rate measuring method includes acquiring a facial image comprising a face of a user, calculating a color signal from a predetermined number of skin regions of interest in the face of the user in the facial image, calculating a pulse wave signal or a respiratory signal by converting an extracted signal into a time domain after extracting a signal in a frequency band corresponding to pulse or respiration in frequency data obtained by converting the color signal into a frequency domain, and measuring a pulse rate or a respiratory rate corresponding to the frequency band based on peak interval information in the pulse wave signal or the respiratory signal.

The measuring of the pulse rate or the respiratory rate may include calculating an average value of peak intervals of pulse or respiration based on the peak interval information, and measuring a pulse rate or a respiratory rate based on the average value of the pulse or respiration peak intervals.

In the measuring of the pulse rate or the respiratory rate, the pulse rate or the respiratory rate may be measured using a frame per second (FPS) of the image and an average value calculated from the pulse wave signal or the respiratory signal.

The calculating of the color signal from the skin regions of interest may include for each of the skin regions of interest, converting an RGB color system of a corresponding one of the predetermined number of skin regions of interest into YC_{g}Cₒ and YC_{b}Cᵣ color systems, calculating a weighted average value of C_{g} and C_{b} color data included in the YC_{g}Cₒ and YC_{b}Cᵣ color systems, and calculating the color signal using the weighted average value of C_{g} and C_{b} color data.

The calculating of the pulse wave signal or the respiratory signal may include calculating the frequency data based on fast Fourier transform (FFT) of the color signal, and calculating the pulse wave signal or the respiratory signal by applying inverse fast Fourier transform (iFFT) to a frequency domain corresponding to the pulse or respiration related frequency band in the frequency data to convert the frequency domain into a time domain.

The predetermined number of skin regions of interest may be plural, the calculating of the color signal, the calculating of the pulse wave signal and the respiratory signal, and the measuring of the pulse rate or the respiratory rate may be performed for each of the skin regions of interest, and an average pulse rate and an average respiratory rate of the user are calculated from an average value of the pulse rate and the respiratory rate of each of the skin regions of interest.

The pulse rate and respiratory rate measuring method may further include calculating an improved pulse rate or an improved respiratory rate of the user, using a pulse rate-and-respiratory rate regression analysis equation DB based on a pulse rate and respiratory rate DB measured from a facial image which stores pulse rates or respiratory rates calculated for a plurality of users and a pulse rate-and-respiratory rate DB measured by a photoplethysmogram (PPG) device which stores pulse rates or respiratory rates measured by a separate measuring device for the plurality of users and a pulse rate or a respiratory rate of the user.

The calculating of a color signal may include applying zoom focusing to make a size of image M (image width)×N (image height) in order to detect a face from the facial image, setting a guide line with m (image width)×n (image height) (in this case, M>m, N>n) to detect a face from the facial image applied with the zoom focusing, and calculating the color signal after combining a starting point and an ending point of a section where the face detection is not performed in the facial image.

The pulse rate and respiratory rate measuring may include, between the calculating of the pulse wave signal or the respiratory signal and the measuring of the pulse rate or the respiratory rate, calculating auto correlation of the pulse wave signal or the respiratory signal data by applying an auto correlation function (ACF) to the pulse wave signal or the respiratory signal. In the measuring of the pulse rate or the respiratory rate, the pulse rate or the respiratory rate may be measured using a sample position of a maximum peak detected from the auto correlation and a next peak.

A non-transitory computer-readable storage medium storing instructions that, when executed by a processor, cause the processor to perform the method above.

In another general aspect, a pulse rate and respiratory rate measuring apparatus using a facial image includes an acquirer, a calculator, and a measurer. The acquirer is configured to acquire a facial image comprising a face of a user. The calculator is configured to calculate a color signal from a predetermined number of skin regions of interest in the face of the user in the facial image, extract a signal of a frequency band corresponding to the pulse or the respiration from frequency data obtained by converting the color signal into a frequency domain, and convert the extracted signal into a time domain to calculate a pulse wave signal or a respiratory signal. The measurer is configured to measure a pulse rate or a respiratory rate corresponding to the frequency band based on peak interval information in the pulse wave signal or the respiratory signal.

The measurer may be further configured to calculate an average value of a peak interval of a pulse or respiration using the peak interval information, and measure the pulse rate or the respiration rate using an average value of the peak interval of the pulse or the respiration.

The measurer may be further configured to measure the pulse rate or the respiratory rate using a frame per second (FPS) of the image and the average value of the peak interval calculated from the pulse wave signal or the respiratory signal.

The calculator may be further configured to change the RGB color system of the predetermined number of skin regions of interest, for each of the skin regions of interest included in the facial image, to YC_{g}Cₒ and YC_{b}Cᵣ color systems, calculate a weighted average value of C_{g} and C_{b} color data included in the YC_{g}Cₒ and YC_{b}Cᵣ color systems, and calculate the color signal using the weighted average value of the C_{g} and C_{b} color data.

The calculator may be further configured to generate the frequency data by calculating fast Fourier transform (FFT) for the color signal, apply inverse fast Fourier transform (iFFT) to a frequency domain corresponding to a pulse or respiration related frequency band in the frequency data to convert the frequency domain into the time domain, to calculate the pulse wave signal or the respiratory signal.

The predetermined number of skin regions of interest may be plural, the calculator and the measurer may measure the pulse rate or the respiratory rate for each of the skin regions of interest, and an average pulse rate and an average respiratory rate of the user may be calculated from an average value of the pulse rate and the respiratory rate of each of the skin regions of interest.

The measurer may be further configured to calculate an improved pulse rate or an improved respiratory rate of the user, use a pulse rate-and-respiratory rate regression analysis equation DB based on a pulse rate and respiratory rate DB measured from a facial image which stores pulse rates or respiratory rates calculated for a plurality of users and a pulse rate-and-respiratory rate DB measured by a photoplethysmogram (PPG) device which stores pulse rates or respiratory rates measured by a separate measuring device for the plurality of users and a pulse rate or a respiratory rate of the user.

The calculator may be further configured to apply zoom focusing to a size of the image to be M (image width)×N (image height) to detect a face from the facial image, perform face detection by setting a guide line with m (image width)×n (image height) (in this case, M>m, N>n) to detect a face from the facial image applied with the zoom focusing, and combine a starting point and an ending point of a section where the face detection is not performed in the facial image, and calculate the color signal.

The calculator may be further configured to calculate auto correlation of the pulse wave signal or the respiratory signal data by applying an auto correlation function (ACF) to the pulse wave signal or the respiratory signal, and the measurer is further configured to measure the pulse rate or the respiratory rate using a sample position of a maximum peak detected from the auto correlation and a next peak.

Other features and aspects will be apparent from the following detailed description, the drawings, and the claims.

### BRIEF DESCRIPTION OF DRAWINGS

FIG. 1 is a flowchart explaining an example of a pulse rate or a respiratory rate measuring method using a facial image according to an embodiment of the present disclosure.

FIG. 2 is a block diagram of an example of a pulse rate or a respiratory rate measuring apparatus using a facial image according to an embodiment of the present disclosure.

FIG. 3 is a view explaining an example of a pulse rate or a respiratory rate measuring process using a facial image according to an embodiment of the present disclosure.

FIG. 4 is a view explaining an example of a method of calculating a pulse rate-and-respiratory rate regression analysis equation DB according to an embodiment of the present disclosure.

FIG. 5 is a view explaining an example of a method of calculating a robust pulse rate-and-respiratory rate using a pulse rate-and-respiratory rate regression analysis equation DB according to an embodiment of the present disclosure.

FIG. 6 is a view explaining an example of a method of calculating a robust pulse rate-and-respiratory rate using a zoom camera according to an embodiment of the present disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

The features of the examples described herein may be combined in various ways as will be apparent after an understanding of the disclosure of this application. Further, although the examples described herein have a variety of configurations, other configurations are possible as will be apparent after an understanding of the disclosure of this application.

An object of the present disclosure is to provide a method and an apparatus for precisely measuring a pulse rate and a respiratory rate of a user using a change in a peak interval of a pulse wave signal and a respiration signal calculated by applying fast Fourier transform (FFT) to a color signal calculated from a predetermined number of skin regions of interest in a face of the user and applying inverse fast Fourier transform (iFFT) to a pulse rate related frequency band (0.6 to 3.5 Hz) and a respiration related frequency band (0.13 to 0.4 Hz), using an image obtained by capturing the face of the user.

Further, another object of the present disclosure is to provide a method and an apparatus for measuring a pulse rate and a respiratory rate of a user by averaging pulse rates and respiratory rates calculated in a plurality of skin regions of interest or more precisely measuring a pulse rate and a respiratory rate of the user using a regression analysis technique.

In order to achieve the above-described objects, according to an aspect of the present disclosure, a pulse rate and respiratory rate measuring method using a facial image includes acquiring a facial image which is an image including a face of a user; calculating a color signal from a predetermined number of skin regions of interest in the face of the user in the facial image; calculating a pulse wave signal or a respiratory signal by converting an extracted signal into a time domain after extracting a signal in a frequency band corresponding to pulse or respiration in frequency data which is data obtained by converting the color signal into a frequency domain; and measuring a pulse rate or a respiratory rate corresponding to the frequency band using peak interval information which is information about an interval of peaks in the pulse wave signal or the respiratory signal.

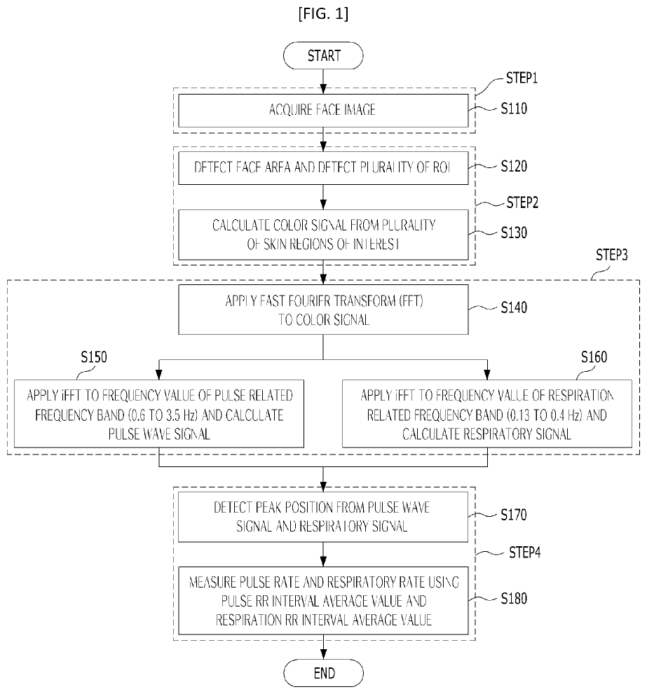

FIG. 1 is a flowchart explaining an example of a pulse rate or a respiratory rate measuring method using a facial image according to an embodiment of the present disclosure.

First (OPERATION 1), a measuring apparatus acquires a facial image, which is an image including a face of a user.

In operation S110, the measuring apparatus acquires a facial image.

Here, the measuring apparatus may acquire an image including a facial skin of a user captured using a camera included therein, an external general camera, an IR camera, or a zoom camera. In this case, the image including the face of the user may refer to a moving image in which the face of the user continuously appears in the same position or continuous photographs with a predetermined time interval. For example, when the measuring apparatus is mounted in a smartphone, an image obtained by capturing the face of a user using the smartphone may also be acquired.

In addition, the measuring apparatus may perform a pre-processing task to detect the face of the user or skin color from the facial image. For example, the measuring apparatus may use a detection model such as Haar cascade, Histogram of Oriented Gradients (HOG), Single Shot Multibox Detector (SSD), You Only Look Once (YOLO) v3 to detect a face area from the facial image and change a setting value (limit a detection area, change an input size, or set a threshold value) and select an appropriate detection model in accordance with the environment of the captured image.

To be more specific, each model shows a variation in detection performance in accordance with a change in an illumination environment. When the face is detected in a poor environment, the detection performance of the YOLO model is the highest, and a skin region of interest may be set in the detected face area.

Second (OPERATION 2), the measuring apparatus calculates a color signal from each of a predetermined number of skin regions of interest in the user's face in the facial image.

In operation S120, the measuring apparatus detects the face area of the user from the facial image and detects a predetermined number of skin regions of interest (ROI).

In this case, the skin region of interest may be a region on the face skin of the user having an arbitrary shape, for example, may be a rectangular shape or a circular shape. Further, the number of skin regions of interest may be one or more and may be a predetermined number.

In operation S130, the measuring apparatus converts the RGB color system of the predetermined number of skin regions of interest in the user's face in the facial image into YC_{g}Cₒ and YC_{b}Cᵣ color systems, calculates a weighted average value of C_{g} and Cₒ color data included in the YC_{g}Cₒ and YC_{b}Cᵣ color systems, and calculates a color signal using the weighted average value of the C_{g} and C_{b} color data.

That is, the measuring apparatus converts a plurality of images included in a facial image having the RGB color system into YC_{g}Cₒ and YC_{b}Cᵣ color systems, respectively, and determines a pixel value using a weighted average value of C_{g} color data of the YC_{g}Cₒ color system and C_{b} color data of the YC_{b}Cᵣ color system. In this case, the YC_{g}Cₒ color system is a color space configured by a luminance Y, a green color difference C_{g}, and an orange color difference Cₒ and the YC_{b}Cᵣ color system is a color space configured by a luminance Y, a blue color difference C_{b}, and a red color difference Cᵣ.

Further, the measuring apparatus changes the RGB color system of the determined skin regions of interest into YC_{g}Cₒ and YC_{b}Cᵣ color systems, calculates a weighted average value of C_{g} and C_{b} color data included in the YC_{g}Cₒ and YC_{b}Cᵣ color systems, and calculates a color signal using the weighted average value of the C_{g} and C_{b} color data.

Third (OPERATION 3), after extracting a signal in a frequency band corresponding to a pulse or respiration from frequency data, which is data obtained by converting the color signal into the frequency domain, the measuring apparatus converts the extracted signal into a time-domain to calculate a pulse wave signal and a respiratory signal.

In operation S140, the measuring apparatus applies FFT (fast Fourier transform) to the color signal calculated in the skin region of interest, calculates a pulse wave signal by applying iFFT (inverse fast Fourier transform) to a pulse wave related frequency band in frequency data, which is converted to the frequency domain (S150) and calculates a respiratory signal by applying iFFT to a respiratory-related frequency band (S160).

For example, after generating frequency data by applying FFT to the color signal and extracting a signal corresponding to a predetermined frequency band from the frequency data, the measuring apparatus calculates the pulse wave signal and the respiratory signal by applying iFFT.

According to another embodiment, after generating the frequency data by calculating FFT for the color signal, the measuring apparatus applies iFFT to the frequency domain corresponding to a predetermined frequency band in the frequency data to convert the frequency domain into the time domain, thereby calculating the pulse wave signal or the respiratory signal.

For example, the measuring apparatus applies the FFT to the color signal to generate frequency data and applies iFFT to each frequency value of the pulse related frequency band (0.6 to 3.5 Hz) and the respiration related frequency band (0.13 to 0.4 Hz) in the frequency data to calculate the pulse wave signal or the respiratory signal.

Finally (OPERATION 4), the measuring apparatus measures a pulse rate and a respiratory rate corresponding to the frequency band using peak interval information, which is information about an interval of peaks in the pulse wave signal or the respiratory signal.

In operation S170, the measuring apparatus detects a peak in the pulse wave signal or the respiratory signal, and in operation S180, it calculates a pulse RR interval average value and a respiratory RR interval average value. The pulse rate and the respiratory rate are measured using the calculated pulse RR interval average value and respiratory RR interval average value.

That is, the measuring apparatus detects a position of the peak of the pulse wave signal or the respiratory signal and calculates the average value of the pulse wave signal or respiratory signal RR intervals using an RR interval (peak-peak interval) calculated from each of the detected peaks. Further, the measuring apparatus may measure the pulse rate or the respiratory rate using the calculated average value.

For example, the measuring apparatus may calculate the number of times of generating a pulse rate or respiratory rate in a predetermined time unit (for example, per minute) using the peak interval information of the pulse rate or respiratory rate.

Pulse rate=PC of Pulse wave signal×60/t

Respiratory rate=PC of Respiratory signal×60/t [Equation 1]

Here, the pulse rate is a pulse rate per minute, and the respiratory rate is a respiratory rate per minute. A peak count (PC) is a peak count detected from the pulse wave signal or the respiratory signal, and t is a total time length (sec) of a signal used for the calculation.

That is, the measuring apparatus detects the position of the pulse wave peak and the respiration peak and calculates an average value of a pulse RR interval and a respiration RR interval using the pulse RR interval (peak-peak interval) and the respiration RR interval (peak-peak interval) calculated from each detected peak. Further, the measuring apparatus may measure the pulse rate and the respiratory rate using the calculated average value of the pulse RR intervals and respiration RR intervals.

At this time, in normal cases, depending on a degree of calmness or excitement, the pulse per minute may be measured to be approximately 40 to 240, and the respiration per minute may be measured to be 8 to 24. Accordingly, a region observed in the frequency domain may be limited to 0.6 Hz to 3.5 Hz for the pulse and 0.13 Hz to 0.4 Hz for the respiration. Further, the range of the respiration and pulse rate related frequency domain may vary depending on a situation, such as a resolution of the image or frame per second (FPS).

According to still another embodiment, the measuring apparatus may measure the pulse rate and the respiratory rate using the frame per second (FPS) of the image and the average value of the peak interval calculated from the pulse wave signal or the respiratory signal.

In this case, the measuring apparatus may measure a robust pulse rate and respiratory rate using the following Equation 2.

Pulse rate=60×Image FPS/(Average value of pulse RR interval (peak-peak interval)

Respiratory rate=60×Image FPS/(Average value of respiration RR interval (peak-peak interval) [Equation 2]

Here, the pulse rate is a pulse rate per minute, and the respiratory rate is a respiratory rate per minute. The image FPS is a frame per second of the facial image, and the average value of the pulse RR intervals and the average value of the respiration RR intervals are average values of the intervals between a peak detected from the pulse signal and the respiration signal and a next peak, respectively.

According to still another embodiment, when the predetermined number of skin regions of interest is plural and the second (OPERATION 2) to final (OPERATION 4) operations are performed for each of the plurality of skin regions of interest, an average pulse rate and an average respiratory rate of the user may be calculated from the averages of the pulse rates and the respiratory rates of the plurality of skin regions of interest.

That is, the measuring apparatus may calculate the pulse rate and the respiratory rate for each of the plurality of skin regions of interest from one image. Further, the measuring apparatus may calculate a robust average pulse rate and a robust average respiratory rate of the user using an average value of the pulse rate and the respiratory rate calculated for the plurality of skin regions of interest.

By doing this, the measuring apparatus may stably measure the pulse rate or the respiratory rate of the user more robustly from a measurement error due to the difference in the illumination.

According to still another embodiment, the measuring apparatus uses a pulse rate and respiratory rate regression analysis equation DB and the pulse rate or the respiratory rate of the user to calculate an improved pulse rate or an improved respiratory rate, which is the improved pulse rate or respiratory rate of the user.

In this case, the pulse rate-and-respiratory rate regression equation DB may be based on a pulse rate and respiratory rate DB measured from the facial image which stores the pulse rate or the respiratory rate calculated for the plurality of users and a pulse rate-and-respiratory rate DB measured by a PPG device which stores a pulse rate or a respiratory rate measured using a separate measurement device for the plurality of users.

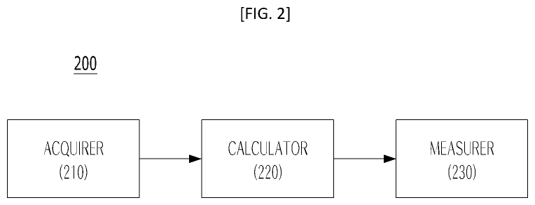

FIG. 2 is a block diagram of a pulse rate and respiratory rate measuring apparatus using a facial image according to an embodiment of the present disclosure.

Referring to FIG. 2, the pulse rate and respiratory rate measuring apparatus 200 using a facial image according to an embodiment of the present disclosure includes an acquirer 210, a calculator 220, and a measurer 230.

In this case, the pulse rate and respiratory rate measuring apparatus 200 using a facial image according to an embodiment of the present disclosure may be mounted in smartphones, tablet PCs, wearable apparatuses, notebook PCs, desktop PCs, and the like.

The acquirer 210 acquires a facial image, which is an image including a face of the user.

The calculator 220 calculates a color signal from a predetermined number of skin regions of interest in the face of the user in the facial image, extracts a signal of a frequency band corresponding to the pulse or the respiration from frequency data, which is data obtained by converting the color signal into a frequency domain and then converts the extracted signal into a time-domain to calculate a pulse wave signal or a respiratory signal.

According to another embodiment, the calculator 220 changes the RGB color system of the predetermined number of skin regions of interest, for each of the plurality of skin regions of interest included in the facial image, to the YC_{g}Cₒ and YC_{b}Cᵣ color systems, calculates a weighted average value of C_{g} and C_{b} color data included in the YC_{g}Cₒ and YC_{b}Cᵣ color systems, and calculates a color signal using the weighted average value of the C_{g} and C_{b} color data.

According to still another embodiment, the calculator 230 generates the frequency data by calculating FFT for the color signal, applies iFFT to the frequency domain corresponding to a pulse or respiration related frequency band in the frequency data to convert the frequency domain into the time domain, thereby calculating the pulse wave signal or the respiratory signal.

Finally, the measurer 230 measures a pulse rate or a respiratory rate corresponding to the frequency band using peak interval information, which is information about an interval of peaks in the pulse wave signal or the respiratory signal.

According to still another embodiment, the measurer 230 may measure the pulse rate or the respiratory rate using the frame per second (FPS) of the image and the average value of the peak interval calculated from the pulse wave signal or the respiratory signal.

According to still another embodiment, the predetermined number of skin regions of interest is plural, the calculator 220 and the measurer 230 measure a pulse rate or a respiratory rate for each of the plurality of skin regions of interest and an average pulse rate and an average respiratory rate of the user may be calculated from the average value of the pulse rate or the respiratory rate of each of the plurality of skin regions of interest.

According to still another embodiment, the measurer 230 may calculate an improved pulse rate or an improved respiratory rate, which is an improved pulse rate or respiratory rate of the user, using a pulse rate-and-respiratory rate regression analysis equation DB based on a pulse rate and respiratory rate DB measured from the facial image which stores pulse rate or respiratory rate data calculated for the plurality of users and the pulse rate-and-respiratory rate DB measured by the PPG device which stores the pulse rate or respiratory rate data measured by a separate measuring device for the plurality of users and the pulse rate or the respiratory rate of the user.

According to still another embodiment, the calculator 220 applies zoom focusing to a size of the image to be M (image width)×N (image height) to detect a face from the facial image, performs face detection by setting a guide line with m (image width)×n (image height) (in this case, M>m, N>n) to detect a face from the facial image applied with the zoom focusing, and combines a starting point and an ending point of a section where the face detection is not performed in the facial image, and then calculates a color signal.

According to still another embodiment, the calculator 220 applies auto correlation function (ACF) to the pulse wave signal or the respiratory signal to calculate an auto correlation of the pulse wave signal or the respiratory signal data, and the measurer 230 measures the pulse rate or the respiratory rate using a sample position of a maximum peak detected from the auto correlation and a next peak.

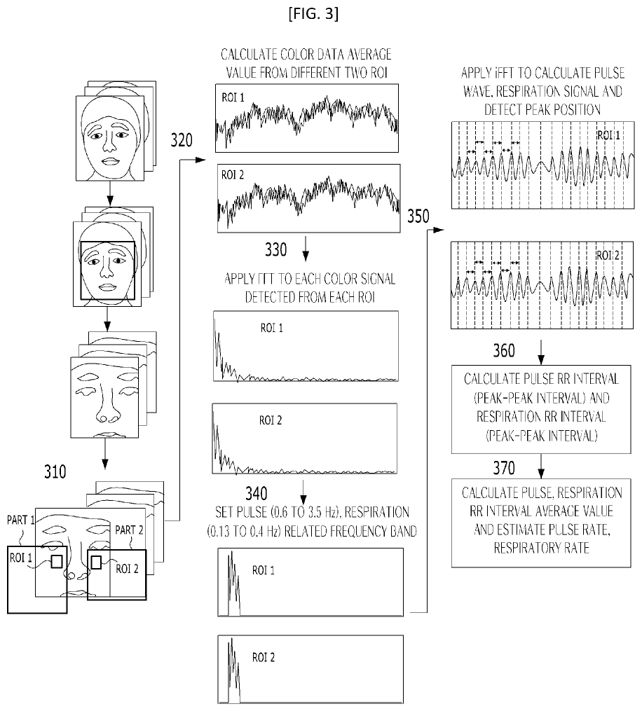

FIG. 3 is a view for explaining a pulse rate or a respiratory rate measuring process using a facial image according to an embodiment of the present disclosure.

The measuring apparatus may acquire an image including a facial skin of a user captured using a camera included therein, an external general camera, an IR camera, or a zoom camera. In this case, the image including the face of the user may refer to a moving image in which the face of the user continuously appears in the same position or continuous photographs with a predetermined time interval. For example, when the measuring apparatus is mounted in a smartphone, an image obtained by capturing the face of a user using the smartphone may also be acquired.

The skin region of interest may be a region on the face skin of the user having an arbitrary shape, for example, may be a rectangular shape or a circular shape. Further, the number of points of interest may be one or more and may be a predetermined number (310).

Further, the measuring apparatus converts a plurality of images included in a facial image having the RGB color system into YC_{g}Cₒ and YC_{b}Cᵣ color systems, respectively, and calculates a color signal using a weighted average value of C_{g} color data of the YC_{g}Cₒ color system and C_{b} color data of the YC_{b}Cᵣ color system. In this case, the YC_{g}Cₒ color system is a color space configured by a luminance Y, a green color difference C_{g}, and an orange color difference Cₒ and the YC_{b}Cᵣ color system is a color space configured by a luminance Y, a blue color difference C_{b}, and a red color difference Cᵣ.

Further, the measuring apparatus changes the RGB color system of the plurality of determined skin regions of interest into YC_{g}Cₒ and YC_{b}Cᵣ color systems, calculates a weighted average value of C_{g} and C_{b} color data included in the YC_{g}Cₒ and YC_{b}Cᵣ color systems, and calculates a color signal using the weighted average value of the C_{g} and C_{b} color data (320).

The measuring apparatus applies the FFT to the color signal calculated in the skin region of interest (330), sets a pulse related frequency band (0.6 to 3.5 Hz) and a respiration related frequency band (0.13 to 0.4 Hz) in frequency data, which is data obtained by converting to the frequency domain (340), applies iFFT to the frequency value of the set band to calculate a pulse wave signal and applies iFFT to the respiration related frequency band (0.13 to 0.4 Hz) to calculate the respiratory signal (350).

The measuring apparatus detects a position of a peak from the calculated pulse wave, respiration signals (350) and calculates a pulse RR interval and a respiratory RR interval value (360).

In addition, the apparatus detects a peak represented in the pulse wave signal and the respiratory signal and calculates a pulse RR interval average value and a respiration RR interval average value. The pulse rate and the respiratory rate are measured using the calculated pulse RR interval average value and respiratory RR interval average value (370).

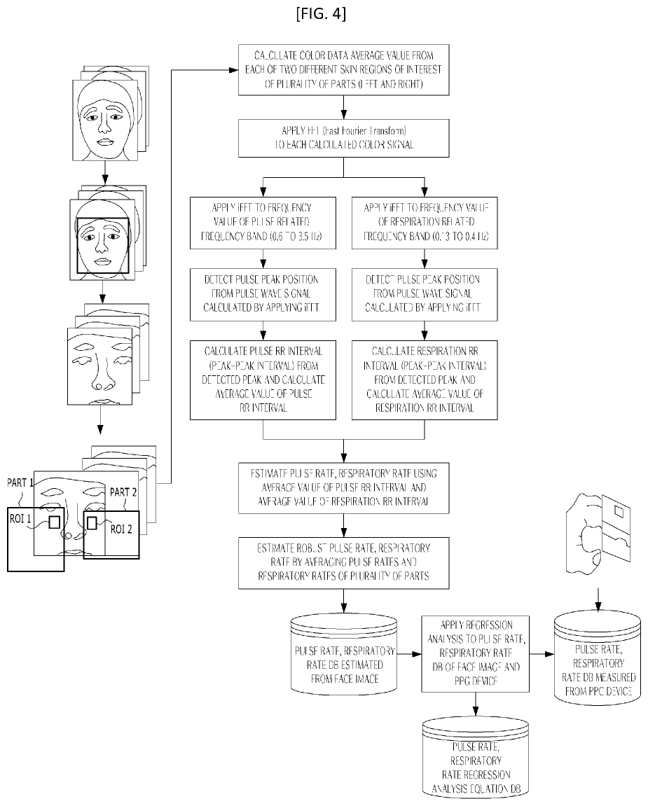

FIG. 4 is a view for explaining a method of calculating a pulse rate-and-respiratory rate regression analysis equation DB according to an embodiment of the present disclosure.

The measuring apparatus calculates the pulse rate and the respiratory rate for a region 1 located on a right cheek of the user and a region 2 located on a left cheek and then calculates a robust pulse rate and respiratory rate using an average value thereof.

The pulse rate and the respiratory rate measured in the facial image of the user are stored in “pulse rate-and-respiratory rate DB measured from facial image” and data measured by a separate measuring device may be stored in a “pulse rate-and-respiratory rate DB measured by a photoplethysmogram (PPG) device.” In this case, regression analysis is applied to two DBs to calculate a regression line (or curve) equation, and the result may be stored in the “pulse rate-and-respiratory rate regression analysis equation DB.”

The measuring apparatus may calculate improved pulse rate and respiratory rate using the pulse rate-and-respiratory rate regression analysis equation DB, and the pulse rate and respiratory rate calculated from the skin region of interest of the user.

A relationship between two variables is represented by a straight line obtained by representing a set of points on a scatter diagram as a straight line, and in the present disclosure, the regression line equation may be derived using the “pulse rate and respiratory rate DB measured from facial image” and the “pulse rate and respiratory rate DB measured by the PPG device.” The regression line equation is represented by Equation 3.

μ=ax+b [Equation 3]

Here, y represents an improved pulse rate or respiratory rate, and x represents a pulse rate or respiratory rate measured from the facial image. In a result obtained by applying actual data, constants a and b may vary depending on used data.

A relationship between two variables is represented by a curved line obtained by representing a set of points on a scatter diagram as a curved line, and in the present disclosure, the regression curve equation may be derived using the “pulse rate-and-respiratory rate DB measured from facial image” and the “pulse rate-and-respiratory rate DB measured by the PPG device.” The regression curve equation is represented by Equation 4.

y=ax²+bx+c [Equation 4]

Here, y represents an improved pulse rate or respiratory rate, and x represents a pulse rate or respiratory rate measured from the facial image. In a result obtained by applying actual data, constants a, b, and c may vary depending on used data.

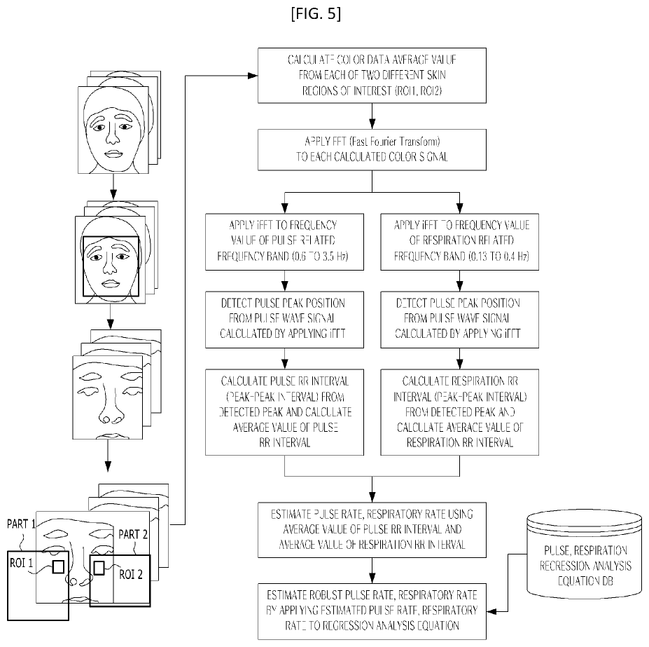

FIG. 5 is a view for explaining a method of calculating a robust pulse rate-and-respiratory rate using a pulse rate-and-respiratory rate regression analysis equation DB according to an embodiment of the present disclosure.

The measuring apparatus may acquire an image including a facial skin of a user captured using a camera included therein, an external general camera, an R camera, or a zoom camera. In this case, the image including the face of the user may refer to a moving image in which the face of the user continuously appears in the same position or continuous photographs with a predetermined time interval. For example, when the measuring apparatus is mounted in a smartphone, an image obtained by capturing the face of a user using the smartphone may also be acquired.

The skin region of interest may be a region on the face skin of the user having an arbitrary shape, for example, may be a rectangular shape or a circular shape. Further, the number of points of interest may be one or more and may be a predetermined number.

The measuring apparatus detects a position of a peak from the calculated pulse wave, respiration signals, and calculates a pulse RR interval and a respiratory RR interval value.

In addition, the measuring apparatus detects a peak represented in the pulse wave signal and the respiratory signal and calculates a pulse RR interval average value and a respiration RR interval average value. The pulse rate and the respiratory rate are measured using the calculated pulse RR interval average value and respiratory RR interval average value.

The measuring apparatus may calculate improved pulse rate and respiratory rate by applying the pulse rate and the respiratory rate calculated in the skin region of interest of the user to the pulse rate-and-respiratory rate regression analysis equation DB.

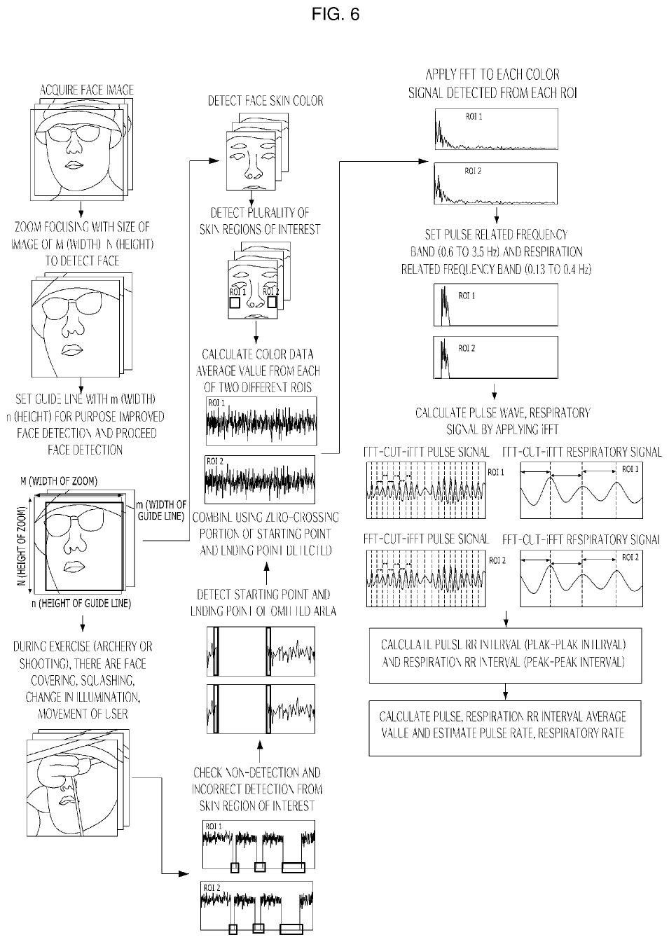

FIG. 6 is a view for explaining a method of calculating a robust pulse rate-and-respiratory rate using a zoom camera according to an embodiment of the present disclosure.

Referring to FIG. 6, the measuring apparatus acquires a facial image, which is an image, including a face of the user from a long distance.

Here, the apparatus may acquire an image including a facial skin of the user photographed using a zoom camera included therein. In this case, the image including the face of the user may refer to a moving image in which the face of the user continuously appears in the same position or continuous photographs with a predetermined time interval. For example, when the measuring apparatus is mounted in a smartphone, an image obtained by capturing the face of a user using the smartphone may also be acquired.

In addition, the measuring apparatus may apply the zoom focusing to the size of the image to be M (image width)×N (image height) in order to detect a face of the user from the facial image.

To be more specific, the zoom has a variation in the detection performance depending on the change of distance, and when optical zoom is used to detect the face in a poor environment, the detection performance may be the highest.

Further, for the purpose of improved face detection, face detection may be performed by setting a guide line with m (image width)×n (image height) for a zoom applied M×N image.

In addition, when the face is partially undetected or incorrectly detected due to the covering or squashing of the face, change in illumination, or movement of the user during exercise (archery or shooting), in order to correct an omitted color signal, a starting point and an ending point of the omitted area are detected and combined using a zero-crossing portion of the detected starting point and ending point to calculate a corrected color signal.

The measuring apparatus may use a detection model such as Haar cascade, Histogram of Oriented Gradients (HOG), Single Shot Multibox Detector (SSD), You Only Look Once (YOLO) v3 to detect a face area from the facial image and change a setting value (limit a detection area, change an input size, or set a threshold value) and select an appropriate detection model in accordance with the environment of the captured image.

To be more specific, each model has a variation in the detection performance depending on the change of illumination environment, and when the face is detected in a poor environment, the detection performance of the YOLO model may be the highest.

In addition, the measuring apparatus detects a face area of the user from the facial age and detects a plurality of regions of interest.

Further, the measuring apparatus converts the RGB color system of the predetermined number of skin regions of interest in the user's face from the facial image into YC_{g}Cₒ and YC_{b}Cᵣ color systems, calculates a weighted average value of C_{g} and C_{b} color data included in the YC_{g}Cₒ and YC_{b}Cᵣ color systems, and calculates a color signal using the weighted average value of the C_{g} and C_{b} color data.

In this case, the skin region of interest may be a region on the face skin of the user having an arbitrary shape, for example, may be a rectangular shape or a circular shape. Further, the number of points of interest may be one or more and may be a predetermined number.

Further, the measuring apparatus changes the RGB color system of the plurality of determined skin regions of interest into YC_{g}Cₒ and YC_{b}Cᵣ color systems, calculates a weighted average value of C_{g} and C_{b} color data included in the YC_{g}Cₒ and YC_{b}Cᵣ color systems, and calculates a color signal using the weighted average value of the C_{g} and C_{b} color data.

The measuring apparatus detects a starting point and an ending point of an omitted area to correct an omitted signal when a signal is undetected or incorrectly detected before applying the FFT to the calculated color signal and calculates a color signal corrected by a combining process using the zero-crossing portion of the detected starting point and the ending point.

In addition, the measuring apparatus applies FFT (fast Fourier transform) to the color signal calculated in the skin region of interest, calculates a pulse wave signal by applying iFFT (inverse fast Fourier transform) to a frequency value of a pulse wave related frequency band (0.6 to 3.5 Hz) of frequency data which is converted to the frequency domain and calculates a respiratory signal by applying iFFT to a respiratory-related frequency band (0.13 to 0.4 Hz).

In addition, the measuring apparatus applies the FFT to the color signal to calculate frequency data and sets the pulse related frequency band (0.6 to 3.5 Hz) and the respiration related frequency band (0.13 to 0.4 Hz) in the frequency data, and then applies iFFT to calculate the pulse wave signal and the respiratory signal.

According to another embodiment, the measuring apparatus calculates the FFT to the color signal calculated in the skin region of interest to calculate frequency data and then applies iFFT to a frequency domain corresponding to the pulse related frequency band (0.6 to 3.5 Hz) and the respiration related frequency band (0.13 to 0.4 Hz) in the frequency data to convert it to the time domain, thereby calculating the pulse wave signal or the respiratory signal.

For example, the measuring apparatus applies the FFT to the color signal calculated in the skin region of interest to calculate frequency data and applies iFFT to each frequency value of the pulse related frequency band (0.6 to 3.5 Hz) and the respiration related frequency band (0.13 to 0.4 Hz) of the frequency data to calculate the pulse wave signal and the respiratory signal.

In addition, the measuring apparatus detects a peak represented in the pulse wave signal and the respiratory signal and calculates a pulse RR interval average value and a respiration RR interval average value. The pulse rate and the respiratory rate are measured using the calculated pulse RR interval average value and respiratory RR interval average value.

That is, the measuring apparatus detects a peak position of the pulse wave signal and the respiratory signal and calculates an average value of the pulse RR intervals and an average value of the respiratory RR intervals using an RR interval (peak-peak interval) calculated from each of the detected peaks. Further, the measuring apparatus may measure the pulse rate and the respiratory rate using the calculated average value.

For example, the measuring apparatus may calculate a pulse rate and a respiratory rate in a predetermined time unit (for example, per minute) using the peak count of the pulse wave signal and the respiratory signal.

The pulse rate and respiratory rate measuring apparatus 200, acquirer 210, calculator 220, and measurer 230 in FIGS. 1-6 that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-6 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access memory (RAM), flash memory, CD-ROMs, CD−Rs, CD+Rs, CD−RWs, CD+RWs, DVD-ROMs, DVD−Rs, DVD+Rs, DVD−RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A pulse rate and respiratory rate measuring method, the method comprising:
acquiring a facial image comprising a face of a user;
calculating a color signal from a predetermined number of skin regions of interest in the face of the user in the facial image;
calculating a pulse wave signal or a respiratory signal by converting an extracted signal into a time domain after extracting a signal in a frequency band corresponding to pulse or respiration in frequency data obtained by converting the color signal into a frequency domain; and
measuring a pulse rate or a respiratory rate corresponding to the frequency band based on peak interval information in the pulse wave signal or the respiratory signal.

2. The pulse rate and respiratory rate measuring method of claim 1, wherein the measuring of the pulse rate or the respiratory rate includes:
calculating an average value of peak intervals of pulse or respiration based on the peak interval information; and
measuring a pulse rate or a respiratory rate based on the average value of the pulse or respiration peak intervals.

3. The pulse rate and respiratory rate measuring method of claim 1, wherein in the measuring of the pulse rate or the respiratory rate, the pulse rate or the respiratory rate is measured using a frame per second (FPS) of the image and an average value calculated from the pulse wave signal or the respiratory signal.

4. The pulse rate and respiratory rate measuring method of claim 1, wherein the calculating of the color signal from the skin regions of interest includes:
for each of the skin regions of interest,
converting an RGB color system of a corresponding one of the predetermined number of skin regions of interest into YC_{g}Cₒ and YC_{b}Cᵣ color systems;
calculating a weighted average value of C_{g} and C_{b} color data included in the YC_{g}Cₒ and YC_{b}Cᵣ color systems; and
calculating the color signal using the weighted average value of C_{g} and C_{b} color data.

5. The pulse rate and respiratory rate measuring method of claim 1, wherein the calculating of the pulse wave signal or the respiratory signal includes:
calculating the frequency data based on fast Fourier transform (FFT) of the color signal; and
calculating the pulse wave signal or the respiratory signal by applying inverse fast Fourier transform (iFFT) to a frequency domain corresponding to the pulse or respiration related frequency band in the frequency data to convert the frequency domain into a time domain.

6. The pulse rate and respiratory rate measuring method of claim 1, wherein the predetermined number of skin regions of interest is plural, the calculating of the color signal, the calculating of the pulse wave signal and the respiratory signal, and the measuring of the pulse rate or the respiratory rate are performed for each of the skin regions of interest, and an average pulse rate and an average respiratory rate of the user are calculated from an average value of the pulse rate and the respiratory rate of each of the skin regions of interest.

7. The pulse rate and respiratory rate measuring method of claim 1, further comprising:
calculating an improved pulse rate or an improved respiratory rate of the user, using a pulse rate-and-respiratory rate regression analysis equation DB based on a pulse rate and respiratory rate DB measured from a facial image which stores pulse rates or respiratory rates calculated for a plurality of users and a pulse rate-and-respiratory rate DB measured by a photoplethysmogram (PPG) device which stores pulse rates or respiratory rates measured by a separate measuring device for the plurality of users and a pulse rate or a respiratory rate of the user.

8. The pulse rate and respiratory rate measuring method of claim 1, wherein the calculating of a color signal includes:
applying zoom focusing to make a size of image M (image width)×N (image height) in order to detect a face from the facial image;
setting a guide line with m (image width)×n (image height) (in this case, M>m, N>n) to detect a face from the facial image applied with the zoom focusing; and
calculating the color signal after combining a starting point and an ending point of a section where the face detection is not performed in the facial image.

9. The pulse rate and respiratory rate measuring method of claim 1, further comprising:
between the calculating of the pulse wave signal or the respiratory signal and the measuring of the pulse rate or the respiratory rate,
calculating auto correlation of the pulse wave signal or the respiratory signal data by applying an auto correlation function (ACF) to the pulse wave signal or the respiratory signal,
wherein in the measuring of the pulse rate or the respiratory rate, the pulse rate or the respiratory rate is measured using a sample position of a maximum peak detected from the auto correlation and a next peak.

10. A non-transitory computer-readable storage medium storing instructions that, when executed by a processor, cause the processor to perform the method of claim 1.

11. A pulse rate and respiratory rate measuring apparatus using a facial image, the apparatus comprising:
an acquirer configured to acquire a facial image comprising a face of a user;
a calculator configured to calculate a color signal from a predetermined number of skin regions of interest in the face of the user in the facial image, extract a signal of a frequency band corresponding to the pulse or the respiration from frequency data obtained by converting the color signal into a frequency domain, and convert the extracted signal into a time domain to calculate a pulse wave signal or a respiratory signal; and
a measurer configured to measure a pulse rate or a respiratory rate corresponding to the frequency band based on peak interval information in the pulse wave signal or the respiratory signal.

12. The pulse rate and respiratory rate measuring apparatus of claim 11, wherein the measurer is further configured to calculate an average value of a peak interval of a pulse or respiration using the peak interval information, and measure the pulse rate or the respiration rate using an average value of the peak interval of the pulse or the respiration.

13. The pulse rate and respiratory rate measuring apparatus of claim 11, wherein the measurer is further configured to measure the pulse rate or the respiratory rate using a frame per second (FPS) of the image and the average value of the peak interval calculated from the pulse wave signal or the respiratory signal.

14. The pulse rate and respiratory rate measuring apparatus of claim 11, wherein the calculator is further configured to change the RGB color system of the predetermined number of skin regions of interest, for each of the skin regions of interest included in the facial image, to YC_{g}Cₒ and YC_{b}Cᵣ color systems, calculate a weighted average value of C_{g} and C_{b} color data included in the YC_{g}Cᵣ and YC_{b}Cᵣ color systems, and calculate the color signal using the weighted average value of the C_{g} and C_{b} color data.

15. The pulse rate and respiratory rate measuring apparatus of claim 11, wherein the calculator is further configured to generate the frequency data by calculating fast Fourier transform (FFT) for the color signal, apply inverse fast Fourier transform (iFFT) to a frequency domain corresponding to a pulse or respiration related frequency band in the frequency data to convert the frequency domain into the time domain, to calculate the pulse wave signal or the respiratory signal.

16. The pulse rate and respiratory rate measuring apparatus of claim 11, wherein the predetermined number of skin regions of interest is plural, the calculator and the measurer measure the pulse rate or the respiratory rate for each of the skin regions of interest, and an average pulse rate and an average respiratory rate of the user are calculated from an average value of the pulse rate and the respiratory rate of each of the skin regions of interest.

17. The pulse rate and respiratory rate measuring apparatus of claim 11, wherein the measurer is further configured to calculate an improved pulse rate or an improved respiratory rate of the user, use a pulse rate-and-respiratory rate regression analysis equation DB based on a pulse rate and respiratory rate DB measured from a facial image which stores pulse rates or respiratory rates calculated for a plurality of users and a pulse rate-and-respiratory rate DB measured by a photoplethysmogram (PPG) device which stores pulse rates or respiratory rates measured by a separate measuring device for the plurality of users and a pulse rate or a respiratory rate of the user.

18. The pulse rate and respiratory rate measuring apparatus of claim 11, wherein the calculator is further configured to apply zoom focusing to a size of the image to be M (image width)×N (image height) to detect a face from the facial image, perform face detection by setting a guide line with m (image width)×n (image height) (in this case, M>m, N>n) to detect a face from the facial image applied with the zoom focusing, and combine a starting point and an ending point of a section where the face detection is not performed in the facial image, and calculate the color signal.

19. The pulse rate and respiratory rate measuring apparatus of claim 11, wherein the calculator is further configured to calculate auto correlation of the pulse wave signal or the respiratory signal data by applying an auto correlation function (ACF) to the pulse wave signal or the respiratory signal, and the measurer is further configured to measure the pulse rate or the respiratory rate using a sample position of a maximum peak detected from the auto correlation and a next peak.
